(19) United States
(12) Patent Application Publication
(10) Pub. No.: US 2009/0196862 A1
(43) Pub. Date: Aug. 6, 2009

(54) High dosage Vitamin D

(76) Inventors: William Davis, Madison, WI (US); Mark A. Nottoli, Lake Bluff, IL (US)

Correspondence Address: PIONEER PATENT, LLC, P. O. BOX 128, NORTHBROOK, IL, 60065-0128, US

(21) Appl. No.: 12/321,655

(22) Filed: Jan. 22, 2009

Related U.S. Application Data
(60) Provisional application No. 61/011,858, filed on Jan. 22, 2008.

(51) Int. Cl. A61K 31/593; A61K 31/122; A61P 35/00; A61P 9/12; A61P 25/24

(52) U.S. Cl. 424/94.1; 514/167; 514/168

(57) ABSTRACT

A preparation containing vitamin D₃ (cholecalciferol), preferably an edible oil, in an amount of 200 IU to 20,000 IU, preferably 1000 IU to 5000 IU, and most preferably about 1500 IU to 3500 IU, with about 2000 IU particularly preferred. Such a preparation is useful in providing adequate nutrition, in the prevention and/or treatment of Seasonal Affective Disorder (SAD), fall prevention, multiple sclerosis, chemoprevention of cancer, accelerated fracture healing, and metabolic syndrome. It acts as an anti-depressant, an anti-hypertensive for renovascular hypertension and high-renin hypertension; and is useful in hyperlipidemia treatment, as well as in the treatment of hypovitaminosis D in the renal failure population. The most important application to the present inventors is in treatment, prevention and also reversal of hypertension in renovascular and high-renin hypertension. The effective amount of vitamin D, based on blood serum content, is equal to or greater than 20 ng/mL, preferably equal to or greater than 40 ng/mL, and most preferably equal to or greater than 50 ng/mL, with no specific upper range, but preferably equal to or less than 200 ng/mL, and more preferably equal to or less than 100 ng/mL.

This utility patent application claims the benefit of the filing date priority provisional U.S. Patent Application No. 61/011,858, filed Jan. 22, 2008.

### FIELD OF THE INVENTION

The present invention concerns high dosage Vitamin D for general health and for the prevention or treatment of various medical conditions. The present invention provides for effective preparations of Vitamin D, of particular benefit is an oil preparation of Vitamin D.

### BACKGROUND OF THE INVENTION

It has long been known that vitamin D helps bones absorb calcium and that rickets marks the worst vitamin D deficiencies. Vitamin D is a fat soluble vitamin that is found in food and is also made in the body after exposure to ultraviolet (UV) rays from the sun. Sunshine is a significant source of vitamin D because UV rays from sunlight trigger vitamin D synthesis in the skin. Vitamin D exists in several forms, each with a different level of activity. Calciferol is the most active form of vitamin D. Other forms are relative inactive in the body. Liver and kidney help convert vitamin D to its active hormone form. Once vitamin D is produces in the skin or consumed in food, it requires chemical conversion in the liver and kidney to form 1,25 dihydroxyvitamin D, the physiologically active form of vitamin D. Active vitamin D functions as a hormone because it sends a message to the intestines to increase the absorption of calcium and phosphorus. Vitamin D is a steroid hormone.

According to the National Institutes of Health Dietary Supplement Fact Sheet for Vitamin D, published May 27, 2004 and updated Aug. 30, 2007, it is thought that the major biologic function of vitamin D is to maintain normal blood levels of calcium and phosphorus. By promoting calcium absorption, vitamin D helps to form and maintain strong bones. Vitamin D also works in concert with a number of other vitamins, minerals and hormones to promote bone mineralization. Without vitamin D, bones can become thin, brittle, or misshapen. Vitamin D sufficiency prevents rickets in children and osteomalacia in adults, two forms of skeletal diseases that weaken bones.

Research in the art also suggest that vitamin D may help maintain a healthy immune system and help regulate cell growth and differentiation.

In the US in the 1930s, rickets was a major public health problem. As a result. the US embarked upon a milk fortification program, wherein 10 micrograms (equivalent to 400 International Units or IU) is added per quart of milk. One cup of vitamin D-fortified milk supplies one-half of the recommended daily intake for adults between the ages of 19 and 50, one-fourth of the recommended daily intake for adults between the ages of 51 and 70, and approximately 15% of the recommended daily intake for adults age 71 and over. Other dairy products, though made from milk, are not presently typically fortified with vitamin D. 1 tablespoon of cod liver oil contains 1,360 IU; 3.5 ounces of cooked salmon contains 360 IU.

Sun exposure is one of the most important sources of vitamin D. However, the season, geographic latitude, time of day, cloud cover, smog and sunscreen affect effective US ray exposure, and hence vitamin D biosynthesis. For example, sunlight exposure from November through February in Boston is insufficient to produce significant vitamin D synthesis in the skin. D.

The Institute of Medicine of the National Academy of Sciences, who recommends dietary intake of nutrients in the Dietary Reference Intakes lists only an Adequate Intake (AI), the amount of vitamin D that should maintain bone health and normal calcium metabolism in healthy people. However, they concluded that there was insufficient scientific information to establish an RDA for vitamin D.

The AIs for vitamin D are listed in the art as either micrograms (μg) or International Unites (IU). The reported biological activity to 1 μg of vitamin D is equal to 40 IUs. As are listed separately for infants, children and adults. For men and women, aged 14-50 years, 200 IU, or 5 μg per day are recommended (including pregnant and lactating women). For ages 51 to 70 years, 400 IU (10 μg) are recommended AIs, and for age 71 and older, the AI is 600 IU (15 μg) per day.

There is a current controversy in the literature regarding whether vitamin D is protective against some cancers (e.g. colon cancer). Although some research suggests its effective, the Institute of Medicine concluded that it is premature to advise anyone to take vitamin D supplements for cancer prevention.

The Institute of Medicine advises that too much vitamin D can cause vitamin D toxicity, leading to nausea, vomiting, poor appetite, constipation, weakness and weight loss. Also, they say that it can raise blood levels of calcium, thought to cause mental status changes such as confusion. High blood levels of calcium can also cause heart rhythm abnormalities and calcinosis (the deposition of calcium and phosphate in the body's soft tissues such as the kidney). The Institute recommends that adult men and women aged 1 to 71 or more years not take more than 2,000 IU (50 μg) per day of vitamin D.

There are limited means in the art for the treatment of atherosclerotic plaques, and no previously known means for reversal of plaque formation. Conjugated linoleic acid (CLA) is which is known to have antiatherogenic effects on its own, reducing the severity of atherosclerotic lesions (Kritchevsky, U.S. Pat. No. 6,555,579, Apr. 29, 2003, for example 1 milligram to 25 milligrams per day for a human adult). There are apparently other beneficial effects of CLA, including reduction of body fat/increase of lean mass; enhancement of bone growth; anticarcinogenic effects, including inhibiting the proliferation of mammary, colorectal, prostate and forestomach cancers; and also some suggestions regarding diabetes and immune response.

There remains a need for the treatment and prevention of a variety of health conditions that are affected by vitamin D, directly or indirectly.

### BRIEF SUMMARY OF THE INVENTION

The present inventors have found that a preparation containing vitamin D₃ (cholecalciferol) provides many health benefits. The preparation containing vitamin D₃ is preferably an edible oil, with gel caps most preferred; tablets of solid vitamin D₃ least preferred; in the range of 200 IU to 20,000 IU, preferably 1000 IU to 5000 IU, and most preferably about 1500 IU to 3500 IU, with about 2000 IU particularly preferred. Such a preparation is useful in providing adequate nutrition, and possibly in the prevention and/or treatment of Seasonal Affective Disorder (SAD), fall prevention, multiple sclerosis, chemoprevention of cancer, accelerated fracture healing, and metabolic syndrome; that it acts as an anti-depressant, an anti-hypertensive for renovascular hypertension and high-renin hypertension; and that it is useful in hyperlipidemia treatment, and treatment of hypovitaminosis D in the renal failure population. The most important application to the present inventors is in treatment, prevention and also reversal of hypertension in renovascular and high-renin hypertension. The effective amount of vitamin D, based on blood serum content, is equal to or greater than 20 ng/mL, preferably equal to or greater than 40 ng/mL, and most preferably equal to or greater than 50 ng/mL, with no specific upper range, but preferably equal to or less than 200 ng/mL, and more preferably equal to or less than 100 ng/mL. These blood serum content levels correlate to the intake of a variety of International Units (or microgram) intakes of the various forms of vitamin D; but in general, the present inventors have found that vitamin D preparations and fortifications on the market are ineffective for reaching the desired blood serum levels. In contrast, the disclosed preparation containing vitamin D₃ is useful.

Other preparations of interest include edible oils containing vitamin D₃ in the amounts listed above in combination with Conjugated Linoleic Acid (CLA), prescription medicines (particularly statins), omega-3 fatty acids, L-arginine, Coenzyme-Q₁₀, Niacin, and other nutrients (ranges of amounts to be described) and vitamin D₃ distributed in the bloodstream from devices such as stents.

### DETAILED DESCRIPTION OF THE INVENTION

One of the present inventor's biggest and most important breakthroughs is that merely by administration of vitamin D to patients who have suffered from the formation of coronary plaques, heart CT scan scores have showed decreased evidence of plaques, amounting to a heretofore unknown reduction in the amount of plaques formed and even an actual reversal in plaques formed.

Calcium is a marker in the blood for atherosclerotic plaque buildup in the arteries; blood calcium levels serves as an indirect gauge of plaque presence. There is a complex interrelationship between vitamin D and calcium metabolism in the body, apparently with a biological cycle relating the two in the bones. Based on this work, that relationship results in an effect in the blood vessels (e.g. in plaques) by vitamin D blood levels.

The present inventors have tried various pharmaceuticals and dietary changes, but the biggest effect has been observed with vitamin D. The dose of Vitamin D that has shown beneficial effect is 10- to several-hundred-fold higher than the once-recommended government recommended daily intake for Vit. D. As the current state of the art, there is no recommended daily intake for vitamin D, and there is controversy over whether there is any beneficial dietary effect from Vit. D supplements.

With regard to the forms of vitamin D in the art administered orally, the present inventor's work showed that ergocalciferol taken orally has not proven to be effective in raising the amount of vitamin D₃ in blood serum. His work also suggests that cholecaliferol taken as the presently commonly available oral solid has little to no effect on vitamin D blood serum levels. Rather, the best form of vitamin D that the present inventors have worked with up to now is an oral oil suspension of vitamin D. The present inventor has observed markedly enhanced bioavailability from vitamin D₃ taken orally in an oil form, as opposed to vitamin D in multivitamins, solid tablet supplements and calcium tablets. A different researcher claims that there is only about a 50% decrease in absorption from the solid versus the oil, but the present inventor has observed far less absorption from the dried solids as opposed to the oil preparation. Furthermore, the present inventor's studies into the effective forms of vitamin D suggest that the public confusion with regard to dietary vitamin D may be a result of the highly variable bioavailability of so-called vitamin D from the various forms of dietary supplementation.

Vitamin D versus Vitamin D precursor. D₂ is ergocalciferol, a Vitamin D precursor. D₃ is cholecalciferol, that is the bioactive form of Vitamin D. D₂ is meant to be a precursor to vitamin D, it supposedly converts in the body to D₃. The dairy industry uses this less expensive form, D₂, to enrich milk and other products with “Vitamin D”. However, with identical IU of D₂ versus IU D₃, D₂ does not lead to as high of an increase of actual vitamin D in blood serum. The present inventor had even used a prescription D₂ preparation (sold as Drisdol® ergocalciferol, Sanofi Withrop Pharmaceuticals, New York), and it led to zero vitamin D₃ in the blood, although the literature reports as high as a 70% conversion of D₂ to D₃ in humans. Sun exposure permits a human body to make its own D₃, but does not affect the conversion of D₂ to D₃.

The dose of vitamin D₃ that is effective for inhibiting additional plaque formation (and even reversing the instance or severity of plaques) is much higher (on the order of thousands of units) than the previously recommended adequate intake (on the order of hundreds of units).

### Delivery System/Dosage Form

Liquid forms of vitamin D₃ include oil, suspension, microencapsulate, gel cap, capsule, injectible, “enriched” liquids (e.g. milk and orange juice); with the oil form being particularly preferred. The biologically active form of the active ingredient is cholecalciferol [D₃=“Vitamin D”], rather than ergocalciferol [D₂, a Vitamin D precursor]. Several companies sell ergocalciferol as “Vitamin D”, although conversion is required to bioactive vitamin D.

In empirical work by the inventor, it was found that the present vitamin D preparation differs significantly from those available in the art in bioavailability. Vitamin D-fortified milk and cereal on the market is typically enriched with ergocalciferol. The presently sold vitamin D supplements are ergocalciferol or cholecalciferol (vitamin D₃) that fail to lead to the blood plasma levels desired by the present inventors.

Solid forms include pills, capsules, multivitamins, “enriched” food (e.g. bread), microencapsulate, capsules, patches, topical creams, and preferably forms of the oil that is a solid (such as microencapsulated, or when another treatment is applied to make it into a powder form.

The dosage form and amount of Vitamin D is related to human blood serum Vit.D levels in that the number of Vit. D International Units adminstered doesn't necessarily reflect how they are absorbed/utilized. Different forms differ in effective bioavailability (e.g. solid versus oil). The effective amount (Vit D IU) for each dosage form for various conditions in humans is related to human blood serum Vit. D levels in that the number of Vit. D International Units (IU) administered doesn't necessarily reflect how they are absorbed/utilized. Different forms differ in effective bioavailability (e.g. solid versus oil). In addition to efficacy for various human conditions, there may be a different level recommended by the present inventors for a nutritional intake for a person without any specific condition.

Also contemplated means for dietary intake of vitamin D₃ is supplemented food, or “medical food”.

Methods of Using Vitamin D₃ for Treatment and/or Prevention
The Conditions for which Vitamin D₃ is Effective

Vitamin D has been suggested in the literature and/or in Dr. Davis' own work to be effective in the treatment and/or prevention of the following human conditions: 1) Seasonal affective disorder is a potentially important area; 2) Fall prevention (was already reported in the literature); 3) Multiple sclerosis (was also already discussed in the literature); 4) Chemoprevention of cancer (esp. breast, bladder, prostate, and colon); 5) Accelerated fracture healing (another potentially important area); 6) Anti-depressant; 7) Anti-hypertensive for renovascular hypertension and high-renin hypertension; 8) Metabolic syndrome; 9) Hyperlipidemia treatment; 10) Hypovitaminosis D in the renal failure population.

Other contemplated medical conditions that may benefit from vitamin D preparations and dosages according to the present invention include boosting of weak immune systems; Cardio-vascular conditions that include CVD prevention; treatment and/or prevention of atherosclerosis/atherosclerotic plaque; aorta specific conditions; and stroke prevention. Additional uses of vitamin D preparations according to the present invention include Diabetes prevention; treatment of Erectile Dysfunction (ED; improved blood flow); and for healthy pregnancy.

### Effective Amounts

### Blood Serum Levels

The effective amount of having an effect may vary from person to person, by race, by gender, by age, from season to season, geographic location of the person and by the condition which is o be treated or prevented, or some other conditions that the patient may have. This disclosure provides outside ranges of recommended vitamin D₃ levels (minimum and maximum) in human blood serum for the various conditions.

In clinical practice with regard to plaque formation, the present inventor observed that blood serum levels of D₃ of about 50 ng/mL provide sufficient effect. Below 50 ng/mL, there is an incremental effect, with a very small effect as low as 20 ng/mL, but a significant effect emerging at 40-45 ng/mL. By 50 ng/mL, there is a significant and often sufficient effect. About 50 ng/mL of 25-hydroxy-vitamin D [25(OH)D] in blood serum provides a reasonably strong effect against plaque buildup.

As to an upper limit of an effective range, it should be noted that some people can get a blood serum level of vitamin D of 80 ng/mL merely through sun exposure (e.g. Costa Rican sun). The upper effective levels are very much more open, without really a true cutoff that has been observed. Merely as a guideline, 100 ng/mL is an upper range that may typically be observed and will still show a beneficial effect, however, lower amounts, such as 80 that may be typical in equatorial/tropical latitudes, or 60 that may be reached with supplements in northern latitudes for sufficient beneficial effects.

According to the absorption/bioavailability from the present inventor's work, it appears that an adequate intake of vitamin D (such as the oil-based D₃ form) is 2000 IU, to lead to the desired blood serum levels of D₃. It should be noted that the previous IA suggested nutritional intake of 200-400 IU, a significantly lower amount than is suggested by the present inventor's work. The present inventor observed one patient who took orally 6000 IU of a powder-based tablet of D₃. That patient's blood serum level was only 15 ng/mL, instead of the 65 ng/mL that would be expected upon taking an equivalent amount (6000 IU) of an oil-based preparation of vitamin D₃.

A suggested range of vitamin D₃ intake to provide beneficial effect is 1000 to 20,000 IU per day of cholecalciferol, preferably in the oil form, rather than a powder/solid form for reversal of atherosclerotic plaques.

The present inventor observed lipid effects (reduced triglycerides, a raise in the levels of HDL [“good cholesterol”] and a reduction in LDL particles [“bad cholesterol”]) from oral supplementation of an oil-based preparation of D₃.

The present inventor observed reductions in inflammatory levels and blood pressure from the administration of oral D₃, which seems to have a positive effect upon the reduction of atherosclerotic plaque, as evidenced by the CT and heart scan “score”, and hence a significant reduction in coronary risk.

### Other Vitamin D Preparations

Also contemplated are combination preparations, for example including 1) Vitamin D together with Conjugated Linoleic Acid (CLA); 2) Vitamin D together with prescription medicines (for example, anti-hypertensives); 3) Vitamin D together with Omega-3 fatty acids; and 4) Vitamin D together with other nutrients (inter alia, calcium).

Contemplated fatty acids for combined preparations with vitamin D according to the present invention includes EPA (eicosapentaenoic acid), DHA (docosahexaenoic acid), DPA (docosapentaenoic acid), and ALA (alpha linolenic acid), with oil sources being from fish or marine, flax seed, krill or algae. Desired amounts of beneficial fatty acids in a typical dosage form are known in the art and need not be enumerated here, but include fatty acid concentration relative ratios (in mg of EPA: DHA per gram of oil): 250:450; 200:600; 500:200; and 400:200.

Contemplated excipients, coloring agents and stabilizers for preparations of vitamin D according to the present invention include those well-known in the art, and need not be specifically enumerated here. However, typical preferred examples include Emulsifiers: Lecithin, bees wax; Stabilizers: vitamin E/tocopherols, ascorbyl palmitate, rosemary extract; and Natural coloring agents (for capsule) e.g. annatto. Nutrients for combination with vitamin D are well-known in the art, and need not be specifically enumerated here. However, typical preferred examples include the following added or emulsified nutrients: (i) Minerals: magnesium, zinc, chromium, calcium, selenium; (ii) Vitamins: beta-carotene, vitamin A, D, E, B-complex; (iii) Nutraceuticals: lutein, zeaxanthin, resveratrol. Sources and amounts per dosage form of these nutrients are well known and include capsule sizes 250 mg, 500 mg, 600 mg, 1000 mg, with nutrient sources including vegetarian, bovine, porcine and dosage forms including chewables.

As an example of the present invention, a preparation of D₃ together with omega-3 fatty acids, has shown excellent results in arterial plaque reduction—so much so that the two appear to be clear-cut/solid backbone nutritional “requirements”. Less required would be L-arginine, Coenzyme Q₁₀ and Niacin.

Also contemplated is the provision of vitamin D₃ on a drug-eluting stent. The technology regarding drug-eluting stents is well-known in the art and need not be enumerated here. A vitamin D-eluting stent addresses the re-stenosis issue, and is an alternative means to introducing vitamin D directly into the blood.

1. A composition comprising vitamin D₃ in an amount ranging from of 200 IU to 20,000 IU.

2. The composition according to claim 1 wherein said composition is in the form of an edible oil.

3. The vitamin D₃ composition according to claim wherein said composition is in the form of a gelatin-coated capsule.

4. The vitamin D₃ composition according to claim 1 wherein said composition is in the form of tablets of solid vitamin D₃

5. The vitamin D₃ composition according to claim 1 wherein said composition contains vitamin D₃ in the range of 1000 IU to 5000 IU.

6. The vitamin D₃ composition according to claim 1 wherein said composition contains vitamin D₃ in the range of about 1500 IU to about 3500 IU

7. The vitamin D₃ composition according to claim 1 wherein said composition contains vitamin D₃ at about 2000 IU.

8. The vitamin D₃ composition according to claim 1, further comprising a conjugated linoleic acid, a statin, an anti-hypertensive, an omega-3 fatty acid, L-arginine, a nutrient, Coenzyme-Q₁₀, or Niacin.

9. The vitamin D₃ composition according to claim 8 that comprises an omega-3 fatty acid.

10. A method for treatment of coronary plaque formation in a mammal comprising administering an effective amount of a vitamin D₃ composition according to claim 1.

11. The use of a vitamin D₃ composition according to claim 1 in a method comprising the step of administering to a mammal an effective amount of said composition for the treatment or prevention of seasonal affective disorder, falls; multiple sclerosis, cancer, bone fractures, depression, hypertension, metabolic syndrome, hyperlipidemia, or hypovitaminosis D.

12. The vitamin D₃ composition according to claim 1 as an active ingredient in a drug-eluting stent.
